(19) United States
(12) Patent Application Publication
(10) Pub. No.: US 2023/0415669 A1
(43) Pub. Date: Dec. 28, 2023

(54) VEHICLE-MOUNTED SLIDING ORNAMENT

(71) Applicant: Kai CHEN, Nanshan Dist (CN)

(72) Inventors: Kai CHEN, Nanshan Dist (CN); Fan YANG, Nanshan Dist (CN)

(21) Appl. No.: 18/337,084

(22) Filed: Jun. 19, 2023

(30) Foreign Application Priority Data
Jun. 23, 2022 (CN) 202221596521.4
Jul. 11, 2022 (CN) 202221775762.5

(51) Int. Cl. B60R 13/00; A61L 9/12

(52) U.S. Cl. CPC B60R 13/005; A61L 9/12

(57) ABSTRACT

The present disclosure relates to the technical field of automotive decoration, and in particular relates to a vehicle-mounted sliding ornament. The vehicle-mounted sliding ornament includes a base provided with a support surface thereon that can be a concave cambered surface; a moving assembly capable of reciprocating on the support surface; and a limit mechanism by which the moving assembly can reciprocate on the support surface along its predetermined trajectory. Preferably, the limit mechanism is a guide rail and its mating components, such that the moving assembly can reciprocate on the support surface along the predetermined trajectory of the rail

### TECHNICAL FIELD

The present disclosure relates to the technical field of automobile decoration, in particular relates to a vehicle-mounted ornament capable of sliding with a car.

### BACKGROUND

In today's society, automobiles have become the main media of transportation in people's daily lives. In our daily life or work, we are used to placing some craft ornaments in the cars. In prior art, some swinging vehicle-mounted sliding ornaments are popular with people. Although the existing vehicle-mounted ornaments are diverse, they look so boring for vehicle personnel on the grounds that many of them are stereotyped; even some of them are difficult to change after being fixed, e.g., those ornaments that use electric energy to rotate/oscillate at a constant frequency; or some of them are always in a fixed state. Therefore, there is an urgent need to provide a vehicle-mounted ornament that can not only swing with the car but also boast interestingness and functionality.

### SUMMARY

In view of the above technical problems, the technical solution of the present disclosure provides a vehicle-mounted ornament capable of freely sliding along with swinging of a car so as to overcome the defects in the prior art.

To realize the foregoing objects, the present disclosure provides the following technical solution:

the present application discloses a vehicle-mounted sliding ornament, comprising a base provided with a support surface thereon; a moving assembly capable of reciprocating on the support surface; and a limit mechanism by which the moving assembly reciprocates on the support surface along its predetermined trajectory.

Further, the support surface is a concave cambered surface, or the support surface is a plane, or the support surface is a continuous combined surface formed from several cambered surfaces and/or planes.

In some embodiments, the limit mechanism comprises a guide rail disposed on the support surface and limits the movement range of the moving assembly on the support surface along a predetermined trajectory of the guide rail.

Further, the limit mechanism further comprises a fixing press part disposed inside the base; a guiding element of which one end is connected to the fixing press part and the other end passes through an opening of the guide rail to be connected to the moving assembly outside the base.

Preferably, the support surface is provided with a slipping component thereon corresponding to its predetermined trajectory, and the slipping component can reduce a friction force between the moving assembly and the contact surface of the moving assembly.

In some embodiments, the moving assembly comprises a sliding support, pulleys and fasteners, the pulleys are mounted on the sliding support through the fasteners, and the fasteners provide fastening and/or support for the pulleys.

Preferably, the fixing press part is further provided with a counterweight block.

The present application has the following advantages as compared with the prior art:

First, the ornament in the present application is provided with a moving assembly that can freely slide. No additional electric energy is needed in the present application, so when a car runs, by means of swaying of the car, the moving assembly is driven to freely slide on the support surface of the base. Second, to provide a better sliding effect, the support surface on the base is designed as a concave cambered surface. When swaying of the car makes the moving assembly swing, the moving assembly can reciprocate in the concave support surface under the effects of multiple forces, including inertia, a centrifugal force, gravitation and the like, greatly improving the interestingness of the ornament. Third, the present application also provides aromatherapy mechanisms so that the present application has an aromatherapy function. Fourth, an integral or independent number plate is provided in the present application, in such a case, phone numbers can be left so that other people are conveniently to contact the car's owner. Fifth, to realize a good ornamental effect, the moving assembly is provided with a decorating part that can be detached and replaced at will.

### BRIEF DESCRIPTION OF THE DRAWINGS

Drawings constituting a part of the present application are used for providing a further understanding to the present disclosure; and schematic embodiments of the present disclosure and descriptions thereof are intended to explain the present disclosure, and should not be construed to unduly limit this invention. In the drawings:

FIG. 1 is an overall structural schematic diagram of an embodiment of an ornament;

FIG. 2 is an overall structural exploded view of an embodiment of the ornament;

FIG. 3 is a structural schematic diagram of a Limit Embodiment 2 of an embodiment of the ornament;

FIG. 4 is a first structural schematic diagram of a Limit Embodiment 3 of an embodiment of the ornament;

FIG. 5 is a second structural schematic diagram of the Limit Embodiment 3 of an embodiment of the ornament;

FIG. 6 is a third structural schematic diagram of the Limit Embodiment 3 of an embodiment of the ornament;

FIG. 7 is a structural schematic diagram of a Limit Embodiment 4 of an embodiment of the ornament;

FIG. 8 is a structural schematic diagram of a Limit Embodiment 5 of an embodiment of the ornament;

FIG. 9 is a structural schematic diagram of a first aromatherapy mechanism of an embodiment of the ornament;

FIG. 10 is a structural schematic diagram of a second aromatherapy mechanism of an embodiment of the ornament.

In the drawings, the identical parts use the identical reference signs. The figures are not necessarily drawn to scale.

The reference signs in the drawings denote: 1—base; 101—support surface; 102—guide rail; 2—moving assembly; 201—sliding support; 202—mounting hole; 203—pulley; 204—fastener; 205—slider; 301—fixing press part; 302—guiding element; 303—pulley assembly; 304—limit slot; 305—limit channel; 306—guide plate; 307—guide hook; 307a—L-shaped guide hook; 307b—T-shaped guide hook; 308—limit rope; 309—first magnetic suction piece; 310—second magnetic suction piece; 4—lens; 5—decorating part; 6—number plate; 71—first aromatherapy mechanism; 7101—aromatherapy stand; 7102—aromatherapy groove; 7103—ventilation window; 7104—aromatherapy tablet; 7201—aromatherapy holder; 72—second aromatherapy mechanism; 7202—opening; 7203—aromatherapy tablet; 7204—through hole; 7205—protrusion.

### DETAILED DESCRIPTION OF THE EMBODIMENTS

The present disclosure will be explained in detail below with reference to the accompanying drawings and embodiments. Each example is provided in the manner of explaining, rather than making limitations to the present disclosure. In fact, those skilled in the art are aware that modifications and variations can be made in the present disclosure without departing from the scope or spirit of the present disclosure. For example, features illustrated or described as part of one embodiment may be used in another embodiment to generate yet another embodiment. Therefore, it is expected that the present disclosure includes such modifications and variations within the scope of the accompanying claims and their equivalents.

In the description of the present disclosure, all directional or positional relationships indicated by the terms including “longitudinal”, “transverse”, “upper”, “lower”, “front”, “back”, “left”, “right”, “vertical”, “horizontal”, “top”, “bottom”, and the like are those based on the drawings, and are merely for the purpose of conveniently describing the present disclosure, instead of requiring the present disclosure to must have the particular orientation or be structured and operated at the particular orientation, and thus cannot be read to limitations to the present disclosure. In the present disclosure, the terms “link”, “connect”, “dispose” and the like should be understood in a broad sense, e.g., fixed connection or detachable connection; or direct connection, or indirect connection via a medium; or cable connection, radio connection, or wireless communication signal connection. For those of ordinary skill in the art, its particular meanings can be understood depending on the particular contexts.

One or more examples of the present disclosure are shown in the appended drawings. The detailed description uses digits and letter signals to refer to the features in the drawings. The identical or similar signs in the drawings and descriptions have been used to refer to the identical or similar parts of the present disclosure. As used in the text, the terms “first”, “second”, “third” and the like are interchangeable so as to distinguish one element from another and are not intended to indicate the position or importance of individual elements.

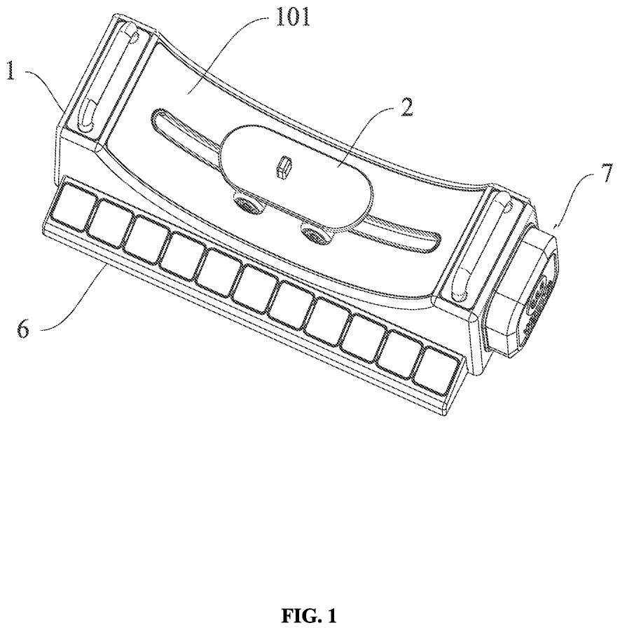

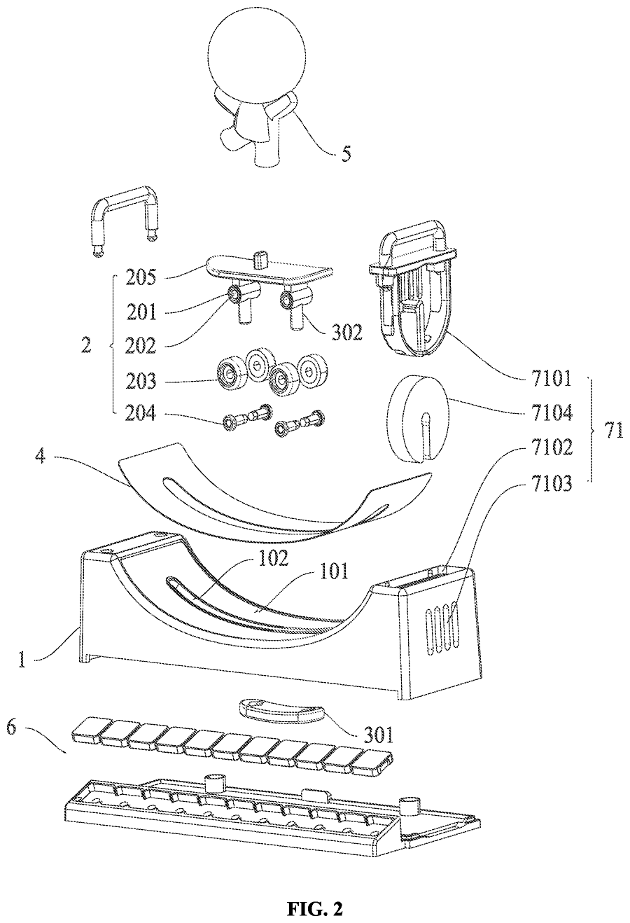

Referring to FIGS. 1-2, the present application designs a vehicle-mounted sliding ornament, which is inspired by a toy skateboard. When we play a skateboard, by use of its small sliding friction, the skateboard can slide forward for a certain distance under the effect of inertia or gravitation so as to reach a good entertainment effect. According to the above principle, the present application designs a vehicle-mounted sliding ornament comprising a base 1 and a moving assembly 2, and the moving assembly 2 is slidably disposed on the base 1 through a limit mechanism. By use of some inertia force in the running process of a car, the moving assembly 2 is enabled to slide on a preset support surface 101 on the base 1, having more sports interestingness.

Wherein, the support surface 101 can be designed into different shapes within a reasonable range. For example, the support surface 101 is a cambered surface, a plane or a continuous combined surface formed from several cambered surfaces and/or planes. In the embodiment, the support surface 101 adopts such a design solution that it is a preferably concave cambered surface, and match of the moving assembly 2 and the base 1 enables the ornament to have an ornamental effect no matter in the perspective of space or plane. The moving assembly 2 is disposed on the support surface 101 in the shape of the concave cambered surface, such that the moving assembly 2 can slide along the support surface 101 in a matched manner with the movement state of a car when the car runs. In a static state, the moving assembly 2 is left in the middle of the base 1 under the effect of own gravitation, having an ornamental value.

In order to increase the visual effect and reduce the friction force of the moving assembly 2 when sliding on the base 1, a slipping component is disposed on the support surface 101 of the base, e.g., a lens 4 in the embodiment is made of glass or a resin material. By use of the lens 4 capable of reflecting light, it will provide an effect like sliding on a water surface when the moving assembly 2 slides, thereby looking so clear and bright and providing quiet and peaceful feeling for people.

To further limit the movement trajectory of the moving assembly 2 and prevent the moving assembly from sliding out of the guide range of the support surface 101 in the sliding process, there requires a limit mechanism. In the present application, limit embodiments are provided as below.

### Limit Embodiment 1

Referring to FIG. 2, the moving assembly 2 comprises a sliding support 201, horizontal mounting holes 202 are formed on the upper part of the sliding support 201, the pulleys 203 are mounted on the sliding support 201 through match of the fasteners 204 and the mounting holes 202, and the fasteners 204 provide fastening and support for the pulleys. A slider 205 is mounted on the top of the sliding support 201 and slides with the sliding support 201. Relative to the slider 205 that slides in a sliding friction manner, the pulleys 203 slide in a rolling friction manner at a small friction coefficient, favoring the slider 205 to slide on the support surface 101.

In some embodiments, preferably, the above pulleys 203 are made of bearings.

The support surface 101 is provided with a guide rail 102 of a strip-shaped hole structure. Preferably, the guide rail 102 can be arranged along the length direction of the support surface 101. A fixing press part 301 is disposed at the lower part of the sliding support 201, and is connected with the sliding support 201 through the guiding element 302 for limiting the moving assembly 2 within the predetermined trajectory range of the guide rail 102. For example, after mounting, the sliding support 201 and the fixing press part 301 are respectively positioned at the inside and outside of the support surface 101, and the fixing press part 301 and a connection structure of the fixing press part connected with the sliding support 201 can effectively prevent the moving assembly 2 from departing from the guide range of the guide rail 102.

In some embodiments, the fixing press part 301 may be a clamping slice on which a clamping groove or a clamping hole is formed to clamp the lower part of the sliding support 201 on the fixing press part 301. Besides, the fixing press part 301 may be in threaded connection with the guiding element 302 at the lower part of the sliding support 201, and when a threaded structure is disposed, an adhesive can be provided for threaded connection to prevent loosening.

In the present application, the moving assembly 2 is of a skateboard structure. Further, in the embodiment, two groups of sliding supports 201, pulleys 203, fasteners 204 and guiding elements 302 are provided, which respectively correspond to the front wheel support and the rear wheel support of the skateboard. Certainly, it is not excluded that there are maybe a plurality of pulleys 203 on the sliding support 201 in some embodiments. For example, two, three, four or more pulleys 203 are provided at the same time. One or more fixing press parts 301 are provided, and their specific quantity can be determined as required.

In some embodiments, when the support surface 101 and the guide rail 102 are set as a slope of an arc shape or with a great radian, to prevent the tail end of the slider 205 from being clamped on the guide rail 102, a counterweight block can be disposed on the fixing press part 301, such that even if the slider 205 goes down the slope, its upper end will not warp to be clamped.

Certainly, the above moving assembly 2 may merely comprise the slider 205, and one end of the above guiding element 302 is connected on the fixing press part 301 and the other end is directly connected with the slider 205. The slider 205 can slide on the support surface 101 under the limit of the fixing press part 301, the guiding element 302 and the guide rail. The above sliding support 201 and the pulleys 203 on the sliding support are disposed to make the moving assembly 2 move more smoothly.

The moving assembly 2 can be designed as the sliding support 201 as well as the pulleys 203 and the slider 205 on the sliding support in the embodiment, designed as merely comprising the slider 205, or designed as merely comprising the decorating part 5 mentioned below.

### Limit Embodiment 2

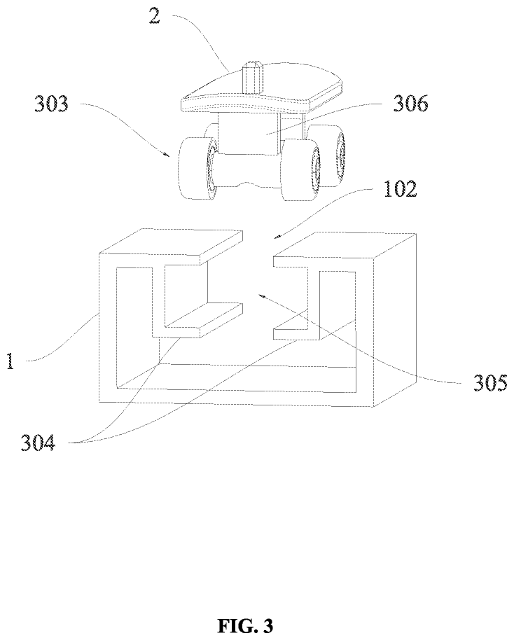

Referring to FIG. 3, the support surface 101 is provided with a guide rail 102 of a strip-shaped hole structure in the embodiment. Preferably, the guide rail 102 can be arranged along the length direction of the support surface 101. The limit mechanism comprises a pulley assembly 303 and a limit slot 304 arranged in the base 1 along the length direction of the support surface 101. The opening of the guide rail 102 is communicated with the interior of the limit slot 304. The limit slot 304 as well as the support surface 101 and the guide rail 102 constitute a limit channel 305, and the pulley assembly 303 is accommodated in the limit channel 305 and can slide along the trajectory of the limit channel 305. The pulley assembly 303 comprises a pulley support and pulleys, and the guide plate 306 is connected with the moving assembly 2 and/or the pulley assembly 303. For example, the guide plate 306 is disposed above the pulley assembly 303, and the guide plate 306 passes through the opening of the guide rail 102 to be connected with the moving assembly 2 outside the base. The moving assembly 2 is the slider 205 in this embodiment. Preferably, the dimension of the guide plate 306 is equal to that of the opening of the guide rail 102 such that the guide plate 306 can slide smoothly when limited on the guide rail 102.

### Limit Embodiment 3

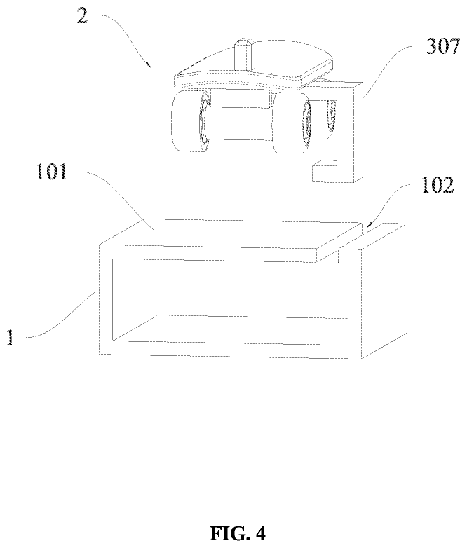

Referring to FIG. 4, in the embodiment, the support surface 101 is provided with a guide rail 102 of a strip-shaped hole structure, and the guide rail 102 is arranged on the top or the side wall of the base 1. The moving assembly 2 in the embodiment comprises a sliding support and pulleys thereon. The limit mechanism comprises a guide hook 307, one end of the guide hook 307 is mounted on the side of the moving assembly 2, the other end is a free end, and the free end passes through the opening of the guide rail 102 to extend into the base 1. The moving assembly 2 is disposed at the outside of the base 1, and is in matched connection with the guide rail 102 through the guide hook 307 so as to guide the moving assembly 2 to slide on the support surface 101 along the predetermined trajectory of the guide rail 102. After mounting, the hook part or end part of the above guide hook 307 is positioned in the base 1, and the pulley assembly 303 is outside the base 1. The guide hook 307 and the opening of the guide rail form sliding fit so as to limit the moving assembly 2 and allow the moving assembly 2 to slide on the support surface 101 along the predetermined trajectory of the guide rail 102.

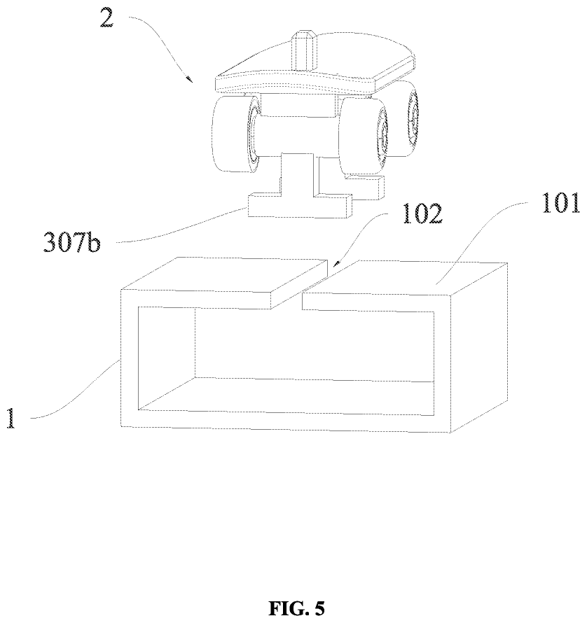

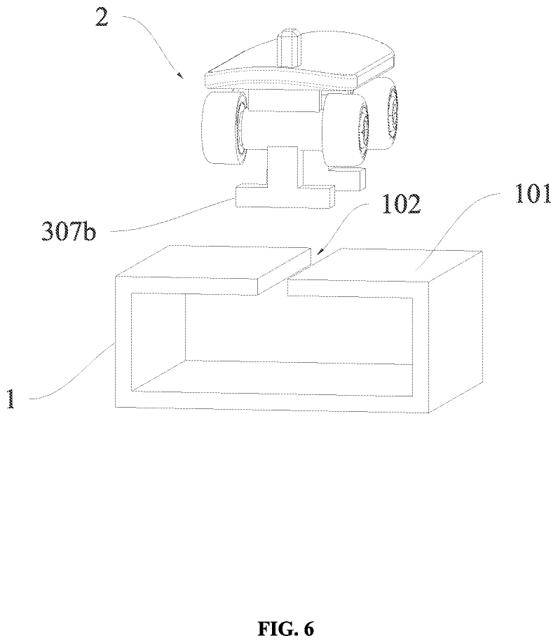

In some embodiments, the end portion of the free end of the above guide hook 307 is L-shaped or T-shaped, e.g., an L-shaped guide hook 307a as shown in FIG. 5 and a T-shaped guide hook 307b as shown in FIG. 6. Generally, the L-shaped guide hook 307a or the T-shaped guide hook 307b is disposed at the bottom of the moving assembly 2.

Besides, the above guide rail 102 is arranged at a middle part or lateral part of the top surface of the base 1 or on a side wall of the base 1. By match of the guide hook 307 in different forms and the guide rail 102 at different positions, the relative position of the moving assembly 2 on the top of the base 1 is changed or adjusted to form different arrangement patterns. For example, the moving assembly 2 is disposed on one side or at the middle part of the base 1 away from a user so as to form different visual effects.

### Limit Embodiment 4

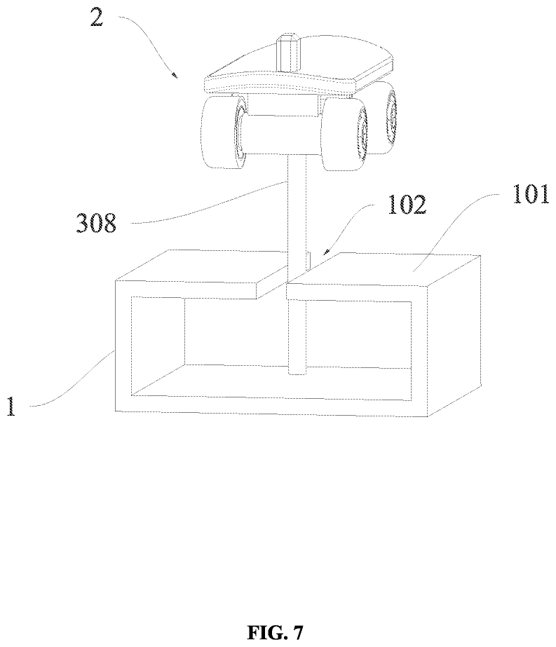

Referring to FIG. 7, the support surface 101 is provided with a guide rail 102 of a strip-shaped hole structure in the embodiment. Preferably, the guide rail 102 can be arranged along the length direction of the support surface 101. The moving assembly 2 in the embodiment comprises a sliding support and pulleys thereon. The limit mechanism comprises a limit rope 308, one end of the limit rope 308 is movably connected in the base 1, and the other end passes through the opening of the guide rail 102 to be connected with the moving assembly 2 outside the base 1. The limit rope 308 is made of a material having a certain elasticity, or may be a common fiber rope. The limit rope 308 is used for limiting and guiding the moving assembly 2 to slide on the support surface 101 along the predetermined trajectory of the guide rail 102.

In some embodiments, the limit rope 308 is made of an elastic material, and one end of the limit rope 308 is movably connected in the base and can slide along the position, corresponding to the opening of the guide rail 102, of the base. For example, a connection end is formed at one end of the limit rope 308, a connection channel corresponding to the position of the opening of the guide rail 102 is formed on the base 1, the connection end of the limit rope 308 goes deep into the connection channel and is connected with the base 1, and the connection end of the limit rope 308 is allowed to slide in the connection channel. Preferably, the position of the connection channel is consistent with the position of an orthographic projection of the opening of the guide rail 102 in the base, such that the limit rope 308 moves in the base 1 with the sliding of the moving assembly 2, and guides and limits the sliding of the moving assembly 2.

### Limit Embodiment 5

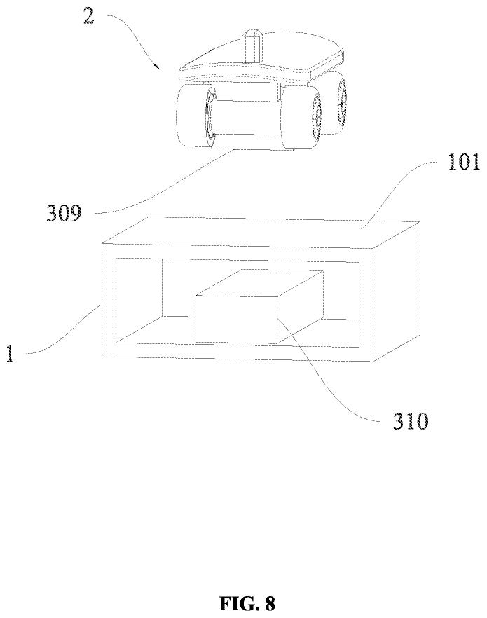

Referring to FIG. 8, the moving assembly 2 in the embodiment comprises a sliding support and pulleys thereon. The limit mechanism comprises a first magnetic suction piece 309 and a second magnetic suction piece 310. The first magnetic suction piece 309 is disposed in the base 1 and arranged along the length direction of the support surface 101, and the second magnetic suction piece 310 is directly or indirectly connected with the moving assembly. For example, the second magnetic suction piece 310 is disposed at the bottom of the moving assembly 2. By magnetic suction match of the first magnetic suction piece 309 and the second magnetic suction piece 310, the moving assembly 2 is limited and enabled to slide on the support surface 101 along the trajectory of the first magnetic suction piece 309.

Both the first magnetic suction piece 309 and the second magnetic suction piece 310 are magnets, or one of them is a magnet and the other one is a part that can be magnetically sucked by the magnet, e.g., an iron part.

Preferably, the support surface 101 is provided with a limit baffle that matches with the first magnetic suction piece 309 and the second magnetic suction piece 310 to limit the moving assembly 2 to slide in the limit baffle. For example, the limit baffle is of a strip-shaped plate structure that protrudes upward from the support surface 101 and extends along the length direction of the support surface 101. Or a pulley groove that is arranged along the length direction of the support surface 101 is formed on the support surface 101, at least one portion of the pulley of the moving assembly is accommodated in the pulley groove to match with the magnetic suction pieces to limit the moving assembly 2 to slide in the pulley groove. In some embodiments, the pulley groove may be of a groove structure that forms a depression on the support surface 101, and the dimension of the pulley groove is slightly greater than that of the pulley so as to facilitate sliding of the pulley in the pulley groove.

In the present application, the above moving assembly 2 is further provided with a decorating part 5 that can be detachably mounted on the moving assembly 2. The decorating part may be a cartoon image, e.g., a bear, a cartoon character, a small car or the like. Preferably, on the base 1, the moving assembly 2 or the decorating part 5, there may be a decoration device. For example, the decoration device is a vibration sensor lamp, so when the car bumps, the vibration sensor lamp automatically lights up.

In the present application, a first aromatherapy mechanism 71 and/or a second aromatherapy mechanism 72 are/is also designed. To be specific, the first aromatherapy mechanism 71 and/or the second aromatherapy mechanism 72 are/is disposed on the base 1 or in the moving assembly 2, or the base 1 and the moving assembly 2 are respectively provided with the first aromatherapy mechanism 71 and/or the second aromatherapy mechanism 72.

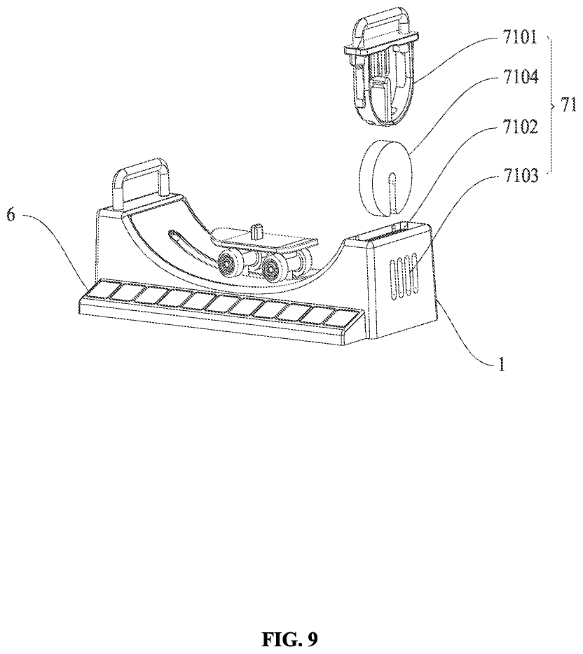

Referring to FIG. 9, in some embodiments, when the first aromatherapy mechanism 71 is disposed on the base 1, the first aromatherapy mechanism 71 comprises an aromatherapy groove 7102 formed in the base 1 and an aromatherapy stand 7101 disposed in the aromatherapy groove 7102, and a ventilation window 7103 is formed on the side wall of the aromatherapy groove 7102. The aromatherapy stand 7101 is provided with a handle at its upper part so that the aromatherapy tablet 7104 is convenient to replace.

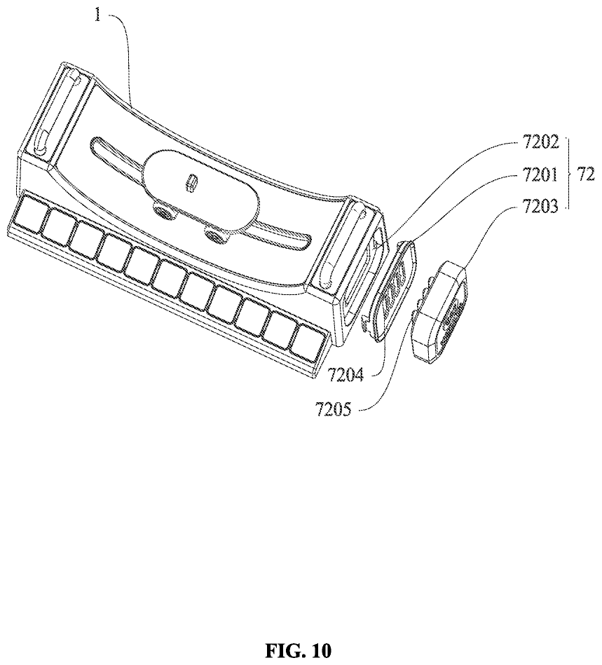

Referring to FIG. 10, in another embodiment, the base 1 is at least provided with a second aromatherapy mechanism 72. The second aromatherapy mechanism 72 comprises an aromatherapy holder 7201 and an aromatherapy tablet 7203, and the aromatherapy holder 7201 is movably mounted on one side of the base 1. In the embodiment, the base 1 is provided with an opening 7202 matched with the aromatherapy holder 7201. One side of the aromatherapy holder 7201 is clamped on the opening 7202, and the other side is connected with the aromatherapy tablet 7203. In the embodiment, the aromatherapy holder 7201 is provided with several through holes 7204, and the aromatherapy tablet 7203 is provided with several protrusions 7205 for match. The aromatherapy tablet 7203 is clamped on the aromatherapy holder 7201 by match of the through holes 7204 and the protrusions 7205. In the embodiment, as the aromatherapy tablet 7203 is corrosive, an independent aromatherapy holder 7201 is adopted in a preferable solution. The aromatherapy holder 7201 is made of a material different from that of the base 1, and the material may be selected from a corresponding corrosion-resistant material in accordance with the characteristics of the aromatherapy tablet 7203 to improve the corrosion resistance of the aromatherapy holder 7201.

Certainly, the above aromatherapy holder 7201 and the base 1 are of an integral design. Only the material of the base 1 can influence the service life of the product.

Besides, as shown in FIG. 1, in the present application, a number plate 6 is further provided. In the embodiment, the number plate 6 is disposed on the base, and a baseplate of the number plate 6 and a baseplate of the base 1 are of an integral design. In some embodiments, the number plate 6 can be separately designed on the vehicle-mounted sliding ornament, such that more unique personalized effects can be collocated.

Although the present disclosure has been described by referring to preferable embodiments, it would be appreciated to make various improvements and equivalent substitutions for parts in the above embodiments without departing from the scope of the present application. In particular, all technical features mentioned in the above embodiments can be arbitrarily combined as long as there are no structural conflicts. The present application is not limited to the particular embodiments disclosed herein, but comprises all technical solutions falling into the protection scope of its claims.

What is claimed is:

1. A vehicle-mounted sliding ornament, comprising:
a base provided with a support surface thereon;
a moving assembly capable of reciprocating on the support surface; and
a limit mechanism by which the moving assembly reciprocates on the support surface along its predetermined trajectory.

2. The vehicle-mounted sliding ornament according to claim 1,
wherein the support surface is a concave cambered surface, or
the support surface is a plane, or
the support surface is a continuous combined surface formed from several cambered surfaces and/or planes.

3. The vehicle-mounted sliding ornament according to claim 1,
wherein the limit mechanism comprises a guide rail disposed on the support surface and limits the movement range of the moving assembly on the support surface along a predetermined trajectory of the guide rail.

4. The vehicle-mounted sliding ornament according to claim 3,
wherein the limit mechanism further comprises
a fixing press part disposed inside the base;
a guiding element of which one end is connected to the fixing press part and the other end passes through an opening of the guide rail to be connected to the moving assembly outside the base.

5. The vehicle-mounted sliding ornament according to claim 4,
wherein the support surface is provided with a slipping component thereon corresponding to its predetermined trajectory, and the slipping component can reduce a friction force between the moving assembly and the contact surface of the moving assembly

6. The vehicle-mounted sliding ornament according to claim 4,
wherein the moving assembly comprises a sliding support, pulleys and fasteners, the pulleys are mounted on the sliding support through the fasteners, and the fasteners provide fastening and/or support for the pulleys.

7. The vehicle-mounted sliding ornament according to claim 4,
wherein the fixing press part is further provided with a counterweight block.

8. The vehicle-mounted sliding ornament according to claim 3, wherein
the limit mechanism further comprises a limit slot disposed in the base and arranged along the trajectory of the guide rail, the limit slot and the support surface constitute a limit channel with which the opening of the guide rail is communicated;
a pulley assembly disposed in the limit channel and movable along the trajectory of the limit channel; and
a guide plate of which one end is connected to the pulley assembly and the other end passes through the opening of the guide rail to be connected with the moving assembly outside the base.

9. The vehicle-mounted sliding ornament according to claim 3,
wherein the limit mechanism further comprises a guide hook of which one end is connected with the moving assembly and the other ends is a free end;
the free end of the guide hook passes through the opening of the guide rail to extend into the base and form sliding fit with the guide rail.

10. The vehicle-mounted sliding ornament according to claim 9,
wherein the free end of the guide hook is L-shaped or T-shaped at an end portion.

11. The vehicle-mounted sliding ornament according to claim 9,
wherein the guide rail is disposed in a middle or lateral part of the top surface of the base or on a side wall of the base.

12. The vehicle-mounted sliding ornament according to claim 3,
wherein the limit mechanism comprises a limit rope of which one end is movably connected in the base, and the other end passes through the opening of the guide rail to be connected with the moving assembly outside the base.

13. The vehicle-mounted sliding ornament according to claim 12,
wherein a connection end is formed at one end of the limit rope, a connection channel corresponding to the position of the opening of the guide rail is formed on the base; the connection end of the limit rope is accommodated in the connection channel, connected with the base and movable in the connection channel;
wherein the position of the connection channel corresponds to the position of an orthographic projection of the opening of the guide rail in the base.

14. The vehicle-mounted sliding ornament according to claim 1,
wherein the limit mechanism comprises a first magnetic suction piece disposed in the base and a second magnetic suction piece disposed on the moving assembly; by match of the first magnetic suction piece and the second magnetic suction piece, the movement range of the moving assembly on the support surface is limited.

15. The vehicle-mounted sliding ornament according to claim 14,
wherein the top surface of the base is provided with a limit baffle that matches with the first magnetic suction piece and the second magnetic suction piece to limit the moving assembly to slide in the limit baffle; or
the moving assembly comprises a sliding support and pulleys on the sliding support, a pulley groove is formed on the support surface, at least one portion of the pulley is accommodated in the pulley groove and matches with the first magnetic suction piece and the second magnetic suction piece to limit the moving assembly to move in the pulley groove.

16. The vehicle-mounted sliding ornament according to claim 1,
wherein the moving assembly is further mounted with a decorating part that can be dismounted repeatedly.

17. The vehicle-mounted sliding ornament according to claim 1,
further comprising at least one second aromatherapy mechanism comprising
an aromatherapy tablet;
an aromatherapy holder of which one side is disposed on the base and the other side is detachably connected with the aromatherapy tablet.

18. The vehicle-mounted sliding ornament according to claim 17,
wherein the aromatherapy holder is provided with several through holes thereon, the aromatherapy tablet is provided with several protrusions for match, and the aromatherapy tablet is detachably mounted onto the aromatherapy holder by match of the through holes and the protrusions;
the base is provided thereon with an opening matched with the aromatherapy holder, and the aromatherapy holder is detachably mounted in the opening, or
the aromatherapy holder and the base are of an integral design.

19. The vehicle-mounted sliding ornament according to claim 1,
further comprising a first aromatherapy mechanism comprising an aromatherapy groove opened on the base, an aromatherapy stand disposed in the aromatherapy groove and an aromatherapy tablet disposed in the aromatherapy stand, and a ventilation window formed on a side wall of the aromatherapy groove.

20. The vehicle-mounted sliding ornament according to claim 1,
further comprising a number plate, wherein the number plate and the base are of an integral design or arranged in a split type.
